(19) United States
(12) Patent Application Publication
(10) Pub. No.: US 2008/0032344 A1
(43) Pub. Date: Feb. 7, 2008

(54) Process for recovery of holocellulose and near-native lignin from biomass

(76) Inventor: John Allan Fallavollita, Edmonton (CA)

Correspondence Address: FULBRIGHT & JAWORSKI, LLP, 666 FIFTH AVE, NEW YORK, NY, 10103-3198, US

(21) Appl. No.: 11/890,113

(22) Filed: Aug. 3, 2007

Related U.S. Application Data
(60) Provisional application No. 60/835,492, filed on Aug. 7, 2006.

(51) Int. Cl. C12P 19/00

(52) U.S. Cl. 435/72

(57) ABSTRACT

A process is provided for the recovery of holocellulose sugars and a near-native lignin co-product from lignocellulosic biomass. The cellulose produced from the process is amenable to subsequent enzymatic hydrolysis to produce monomeric sugar units which can be combined with hemicelluloses-derived sugar units to be co-fermented to produce biofuels and/or chemicals. The process can include either single or multiple hydrothermal treatments of the biomass in aqueous solution under pressure at selected pH and temperature conditions to produce a first liquid phase containing mostly hemicellulose sugars, and a first solid stage containing native lignin. The first solid phase can be subjected to an organosolv treatment to produce a second liquid phase containing most of the near-native lignin as a dissolved component, and a second solid phase containing mostly cellulose. The second liquid phase can be processed to recover near-native lignin powder. The second solid phase can be exposed to hydrolysis enzymes and fermentation yeasts and/or recombinant organisms to produce a biofuel or biochemical. The second solid phase can further be combined with the first liquid phase so as to allow simultaneous saccharification and co-fermentation of the holocellulose-derived sugars.

### TECHNICAL FIELD

The present invention relates generally to a process of refining biomass into individual useful components, more particularly, a process for treating biomass to separately recover holocellulose and near-native lignin therefrom whereby the lignan and holocellulose-derived sugars can then be subjected to different treatments to produce fuels, chemicals, and/or new materials.

### BACKGROUND

Lignocellulosic biomass is the most abundant organic resource on earth. It is commonly referred to as biomass. Biomass includes all plant and plant-derived material such as crops, agricultural food and feed crop residues, wood and wood residues, and industrial and municipal wastes, one such example including waste paper. The three major components of biomass are hemicellulose, lignin, and cellulose. The term “holocellulose” refers to the sum of both hemicellulose and cellulose in the lignocellulosic biomass.

Biomass is a renewable resource with great potential as a sustainable energy source, particularly in view of the limited supply of fossil fuels, rising fuel prices and environmental concerns. Biomass can be refined in a number of ways to produce valuable fuels, chemicals, and materials.

In one method the focus is on a pretreatment that either liberates the cellulose in a form that provides optimum properties for papermaking or chemical production or, alternatively, liberates and alters the cellulose to make it more accessible to enzymes that convert the carbohydrate polymers into fermentable sugars.

For example, in the paper industry, pulping processes have commercially been used for separating cellulose from lignin, hemicelluloses, and other components of lignocellulosic biomass. In these processes, the structurally useful forms of hemicellulose and lignin are largely under-utilized. Only approximately 40% of the biomass is recovered in useable forms in a common Kraft™ pulping process. A major portion of the hemicellulose sugars as well as the structural integrity of native lignin are substantially degraded during this process and report to a black liquor stream that is subsequently burnt.

In another example, the refining of biomass for ethanol production generally is intended to modify the cellulose structure and facilitate its reaction with enzymes to produce monomeric sugar units that are subsequently fermented. In some modifications of this method there is also an emphasis placed on recovering the hemicellulose sugar fraction. In neither case is there any intent to recover lignin as a valuable co-product. Indeed there exists a view that all pretreatment methods be classified only in their ability to cost-effectively produce cellulose that is amenable to enzymatic hydrolysis and fermentation (Mosier et al., 2005). Little or no regard has been placed on the ability to recover lignin in a value-added form in biofuels production.

An approach proposed by U.S. Pat. No. 5,730,837 issued to Black et al. attempts to rectify this situation. The patent discloses a method using a mixture containing an alcohol, water and a water-immiscible ketone to solubilize lignin and hemicellulose, and leave cellulose in a solid pulp phase. The resulting liquid phases comprise a water-immiscible ketone phase containing lignin and an aqueous phase containing dissolved sugars and hemicellulose.

Although Black's method produces cellulose, lignin and hemiceliulose, other byproducts can be found in the aqueous phase such as acetic acid, ketone, alcohol and furfural. These undesirable contaminants may be difficult to separate and refine, particularly in large-scale operations. In addition, the separation of lignin from hemicellulose relies on liquid-liquid separation, which poses certain difficulties and raises costs upon scaling up to larger operations or when a change in the processing parameter is desired.

Accordingly, there is a need for a process that is readily adaptable for continuous operation and large-scale recovery of the majority of sugars in the holocellulose while at the same time providing a lignin product that is structurally similar to native lignin. Also, there is a need for an improved process, employing conventional equipment, for sequentially producing high quality and good yields of holocellulose sugars and a near-native lignin relative to the amount of biomass that is processed.

In particular, there is a need for an efficient fractionation system that minimizes hemicellulose sugar degradation, and recovers lignin and cellulose in useful desirable forms.

### SUMMARY

A process for separately recovering holocellulose and a near-native lignin product from biomass is provided.

In a first stage of the process (“Stage 1”), lignocellulosic biomass can be subjected to one or more hydrothermal treatments to produce a first liquid phase containing hemicellulose-derived sugars, and a first solid phase. The first liquid phase and the first solid phase can then be separated from one another.

In a second stage of the process (“Stage 2”), the first solid phase can be subjected to an organosolv treatment to produce a second liquid phase containing dissolved, near-native lignin and a second solid phase containing mostly cellulose. The second liquid phase and second solid phase can then be separated from one another. The second liquid phase containing near-native lignin can then be subjected to a change in pH, temperature, and/or pressure change to precipitate the dissolved near-native lignin that can then filtered and recovered as a solid powder.

In a third stage of the process (“Stage 3”), the second solid phase containing mostly cellulose can be treated with cellulase enzymes to hydrolyse the crystalline structure to glucose, and can be followed by fermentation of the glucose with yeast and/or an appropriate recombinant organism to produce a biofuel and/or chemical. The second solid phase containing mostly cellulose may also be combined with the hemicellulose-derived sugars from the first liquid phase to allow simultaneous saccharification (of cellulose to glucose) and co-fermentation (of holocellulose-derived sugars) to take place in a single vessel.

The hydrothermal treatment in Stage 1 can utilize heat in an aqueous medium, at a predetermined pH, temperature and pressure, to isolate hemicellulose-derived sugars from the biomass. The organosolv treatment in Stage 2 can utilize at least one organic solvent in water, at a predetermined solvent-to-water ratio, to isolate near-native lignin in a liquid phase and cellulose in a solid phase. The enzymatic hydrolysis of cellulose to produce glucose sugar and the fermentation of glucose in Stage 3 can be carried out in a broth of enzymes, yeast and/or recombinant organisms, solids-to-liquid ratio, and controlled temperature so as to produce a biofuel (e.g., bioethanol and/or biobutanol) and/or a biochemical such as 1,3 propanediol.

In one embodiment, the first liquid phase containing hemicellulose-derived sugars obtained from Stage 1 of the process can be isolated from the lignocellulosic biomass prior to using the organosolv treatment in Stage 2 to recover near-native lignin and cellulose from the first solid stage. This can preserve the structural integrity of the hemicellulose-derived sugars, since these are relatively more susceptible to chemical degradation than either lignin or cellulose. In addition, the hemicellulose is not carried through the entire process and, therefore, its degradation and formation of unintended by-products can be minimized.

In another embodiment, the use of a radially well-mixed, countercurrent solids-liquid flow system for the separation envisaged in Stage 1 can be used as it is known that this arrangement may reduce the amount of undesirable reaction products resulting between acids in the solution and monomeric sugar produced from the hydrolysis of hemicellulose. A countercurrent flow system that has the ability to mix the solids phase in the radial direction in a vigorous manner can be used to reduce the amount of lignin dissolution.

In a further embodiment, the hemicellulose-derived sugars can be isolated in a way that minimizes the degradation of the native lignin in the first solid phase. By properly adjusting the process parameters of time, pH, temperature and pressure, it is possible to achieve a major separation of hemicellulose-derived sugars from the input biomass without severe damage to the structural integrity of the native lignin.

In one embodiment, an efficient process for separating lignocellulosic biomass into holocellulose sugars and near-native lignin that is readily adaptable for large-scale operation is provided.

In another embodiment, an efficient process for separating hemicellulose, lignin, and cellulose from lignocellulosic biomass, while maximizing their recovery and minimizing degradation of the lignin is provided.

In a further embodiment, an efficient process for combining hollocellulose sugars in a vessel to conduct simultaneous saccharification and fermentation to produce a fuel or chemical is provided.

The process described herein can be carried out as a batch process or it can be carried out as a continuous process. The process can produce desirable end products from the biomass that may be further processed. Since the hemicellulose is relatively more susceptible to chemical degradation than lignin or cellulose, the hemicellulose component can be isolated from the lignocellulosic biomass in Stage 1. The hemicellulose-derived sugars can be recovered in a first liquid phase and separated from the first solid phase, prior to an organosolv treatment. Accordingly, the hemicellulose-derived sugars are not carried through the entire process, and degradation and formation of unintended by-products can be minimized.

The organosolv treatment in Stage 2 can utilize organic solvents for enhancing the recovery of near-native lignin in a liquid phase and cellulose in a solid phase. The liquid phase containing near-native lignin can be much easier to separate from the cellulose by any liquid-solid separation technique, thereby minimizing loss during separation and improving the yield of near-native lignin and cellulose.

As evident from the above, both Stage 1 and Stage 2 can produce liquid and solid phases, which can be easily and more efficiently separated using known liquid-solid separation techniques. The liquid-solid phase separation can be more easily adaptable to scaling up for large industrial applications.

The process can also generate base or platform chemicals, namely, hemicellulose and hemicellulose-derived sugars, near-native lignin and cellulose and cellulose-derived sugars, which can be utilized to produce a range of fuels, chemicals, and/or biomaterials, for example, biobutanol; 1,3 propanediol; and near-native lignin resins, respectively.

Broadly stated, a method is provided for separately recovering hemicellulose sugars, lignin and cellulose from biomass, the method comprising the steps of placing biomass in an aqueous environment to form an aqueous biomass mixture; separating a first solid phase and a first liquid phase containing hemicellulose sugars from the aqueous biomass mixture; separating a second solid phase containing cellulose and a second liquid phase containing lignin from the first solid phase; and recovering cellulose from the second solid phase.

Broadly stated, a method is provided for separately recovering hemicellulose sugars, lignin and cellulose from an aqueous biomass mixture, the method comprising the steps of separating a first solid phase and a first liquid phase containing hemicellulose sugars from the aqueous biomass mixture; separating a second solid phase containing cellulose and a second liquid phase containing lignin from the first solid phase; and recovering cellulose from the second solid phase.

Other features and embodiments of the process described herein will become apparent to those skilled in the art from the reading of the following detailed description in view of the accompanying drawings and the appended claims.

### BRIEF DESCRIPTION OF THE DRAWINGS

FIG. 1 is a graph depicting a representation of controlled fractionation kinetics of hemicellulose and lignin of a process for treating biomass.

FIG. 2 is a block diagram depicting a process for treating lignocellulosic biomass to produce a near-native lignin and holocellulose-derived sugars which can be converted to a biofuel and/or a biochemical.

### DETAILED DESCRIPTION OF EMBODIMENTS

In order to promote an understanding and appreciation of a process for recovering holocellulose sugars and near-native lignin from biomass, a number of embodiments thereof now will be described. It will be understood that while certain embodiments are described, all modifications and further utilizations of the principles of these embodiments, as would occur to those ordinarily skilled in the art to which the process relates, are contemplated as being a part of the process.

A process is provided for fractionating lignocellulosic biomass into hemicellulose, near-native lignin and cellulose. The process can comprise a first stage (“Stage 1”) wherein lignocellulosic biomass is placed in an aqueous environment to form an aqueous biomass mixture. The aqueous biomass mixture can be subjected to a hydrothermal treatment to produce a first liquid phase containing hemicellulose-derived sugars, and a first solid phase. The first solid phase and the first liquid phase may then be separated. In a second stage (“Stage 2”), the first solid phase can be subjected to an organosolv treatment that produces a second liquid phase containing near-native lignin and a second solid phase containing mostly cellulose. The second liquid phase and the second solid phase may then be separated. In a third stage (“Stage 3”), the pretreated, solid cellulose is amenable to enzymatic hydrolysis to produce glucose sugar which is then fermented to produce a biofuel and/or biochemical. In addition, the hemicellulose-derived sugars contained in the first liquid phase can be combined with the cellulose contained in the second solid phase in a single reactor to allow simultaneous saccharification (of cellulose to glucose sugar) and co-fermentation of the glucose and hemicellulose-derived sugars to form a biofuel and/or a biochemical.

The particular lignocellulosic material employed as a feedstock for the aqueous biomass mixture is not critical and can be, in one embodiment, derived from a variety of sources, such as plant biomass and cellulosic residues. In another embodiment, biomass that has either equal or higher hemicellulose content than native lignin can be used. Thus, agricultural crop residues such as cereal straws, corn stover, sugarcane bagasse, and grain hull/bran; and dedicated energy crops such as hybrid poplar, switch grass and reeds would likely benefit more from the teachings described herein than a softwood-based biomass such as pine wood.

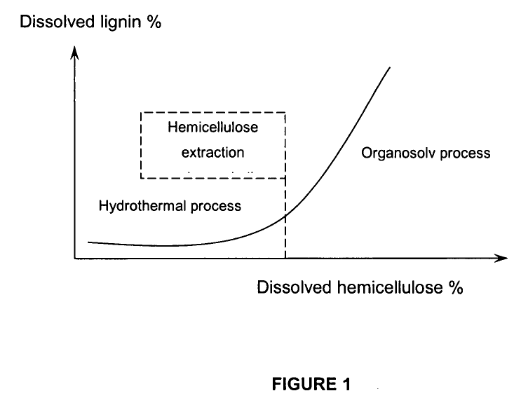

An embodiment of the process is shown graphically in FIG. 1. It involves the determination of the division point between the Stage 1 hydrothermal treatment and the Stage 2 organosolv treatment. Process parameters in the hydrothermal treatment stage—such as reactor geometry and mixing characteristics, temperature, solids/liquid ratio, pH and reaction time—can be chosen in such a way that the hemicellulose extraction from the lignocellulosic biomass can be maximized in Stage 1, while minimizing native lignin dissolution. The organosolv treatment in Stage 2 is designed to maximize lignin extraction while minimizing its degradation and, at the same time, render the cellulose more amenable to enzymatic attack to produce glucose sugar during Stage 3 of the process.

The hydrothermal treatment can comprise treatment in a mostly aqueous environment at condition parameters that include pH, temperature, pressure and time. The pressure maintained in this process step can be generally well above that of atmospheric pressure and sufficient to maintain a mostly liquid phase with little steam production. The hemicellulose component can be recovered from the lignocellulosic biomass into an aqueous phase in differently sized structural units of sugars. These forms of hemicellulose include monomers, oligomers and polymers (i.e. monosaccharides, oligosaccharides, and polysaccharides).

The condition parameters of the hydrothermal treatment determine not only the total amount of hemicellulose recovered, but also the forms of the resulting sugars.

The hydrothermal treatment involves distinct, but complementary, mechanisms that include solubilization and hydrolysis. The contribution of each of these two mechanisms to hemicellulose recovery is highly dependent on the condition parameters.

A wide range of condition parameters can be employed in the hydrothermal treatment stage, which makes the present invention suitable for processing a diverse group of lignocellulosic biomass feedstocks (mentioned above), as well as for producing tailor-made sugar units or forms of hemicellulose to meet specific end uses.

In some embodiments, the pH during the hydrothermal treatment can be generally in the range of about 4 to about 9, and can be adjusted by adding an acid or an alkali. In other embodiments, there is no addition of either alkali or acid as it is well known that an aqueous medium kept under pressure and elevated temperature can be an effective way to hydrolyze hemicellulose.

If pH control is used then an acid may be selected from the group consisting of an inorganic acid and an organic acid. The inorganic acids can include any of the various acids that do not contain carbon atoms, such as sulphuric acid, nitric acid, hydrochloric acid or phosphoric acid. The organic acids can include any of the various acids containing one or more carbon-containing atoms such as acetic acid and carboxylic acid. The alkalis can include, but are not limited to, a carbonate or a hydroxide of an alkali metal such as sodium hydroxide, potassium hydroxide, and sodium carbonate.

If any of one of the abovementioned acids or alkalis are used in the hydrothermal treatment, then care must be exercised to ensure that the concentration of said acid or alkali relative to the amount of biomass is low enough to avoid a significant degradation of the native lignin and the production of undesirable reaction products such as furfural from reactions with hemicellulose-based monomeric sugar.

The hydrothermal treatment can also be autocatalyzed so that a catalyst can be produced naturally during the treatment and, therefore, the addition of an external catalyst is not necessary. For example, the hemicellulose hydrolysis may be catalyzed by acetic acid that is naturally released from the biomass during the hydrothermal treatment.

It has been determined that the pH can play a significant role in determining the yield, composition and form of the recovered hemicellulose. For example, at a pH ranging from about 1 to about 7, acid hydrolysis can be the predominate mechanism for producing monosaccharide forms of hemicellulose.

When production of polysaccharide and/or oligosaccharide forms of hemicellulose is desired, the hydrothermal treatment can be performed under moderate alkaline conditions, where the pH is in the range of about pH 7.5 to about pH 8.0. In the alkaline pH ranges, such as those greater than pH 7, the hemicellulose can be dissolved mainly through the solubilization mechanism.

It should be noted that too high a pH potentially can cause greater hydrolysis of lignin, which is undesirable during the hydrothermal treatment stage. To prevent this, the pH can be kept to about 9 or less.

Where crude plant biomass materials are employed as the biomass feedstock, it has been determined that these materials can have a self-buffering capacity. In addition, some cellulosic materials may have an alkaline pH initially. These naturally occurring properties may be advantageous and can lead to a hydrothermal treatment which is simple and inexpensive, since little or no additional measures of pH control may be necessary. However, where plant biomass materials or microcrystalline cellulose or other biomass materials are employed, it is desirable to initiate pH control. The pH can be monitored using standard equipment.

It is well known that the reactor geometry and mixing characteristics in hydrothermal treatment has a major impact on the dissolution of hemicellulose as well as lignin. For example, if a percolation reactor is used (wherein the biomass is maintained in a fixed bed and liquid water is continuously flowed through the bed), then the degree of hemicellulose dissolution and recovery of xylose, arabinose, and other monomeric five-carbon sugars can be greater than the case where the same biomass is exposed to liquid hot water in a batch reactor where the liquid and solids stay in contact for the entire duration of the reaction.

Unfortunately, the lignin suffers greater degradation in a percolation reactor than in a batch system. Hence, there needs to be a balance in the manner in which the reactor is designed and operated in Stage 1. For commercial systems, one approach is to operate in a countercurrent flow regime using screw-type reactors that have radial mixing of the solids along the full length of the reactor shaft. A combination of this system and a programmed temperature-time protocol that minimizes exposure time to temperatures above 180° C. can lead to the optimum recovery of both lignin and hemicellulose fractions.

In Stage 1, the carbohydrate chain in hemicellulose can also be cleaved by the action of specific enzymes. Similar to the acid hydrolysis (described above), this enzyme-mediated hydrolysis removes sugar units from the hemicellulose, which units are rendered water-soluble and end up in the first liquid phase. The enzyme used can be selective in its site of cleavage and, therefore, produces specific sized sugar units of hemicellulose. This enzyme treatment can be incorporated into the hydrothermal treatment when the pH is in the range from about 4 to about 6. The enzyme-mediated hydrolysis can comprise the use of at least one enzyme including, but not limited to, ferulic acid esterase, xylanase and arabinase.

If enzymes are used in Stage 1, then in one embodiment, the enzymes can be used in conjunction with a hydrothermal treatment. In another embodiment, the enzymes can be added to the liquid fraction produced from Stage 1, which has a high degree of polymers and oligomers present. For example, this can be achieved in a multiple reactor configuration where the hemicellulose is exposed to a first treatment of liquid hot water at lower temperature such as in the range 60-100° C. In this case, the liquid hydrolyzate can contain a higher concentration of oligomers and polymers than monomeric sugar.

During the hydrothermal treatment, the lignocellulosic biomass material can be heated to a temperature within the range of about 60° C. to 220° C. Temperature control can be accomplished in a known manner using standard heating and monitoring equipment as well known to those skilled in the art. For example, the biomass can be suitably heated and maintained by means such as electric heating, steaming or any other suitable means known to those skilled in the art.

The time period of the hydrothermal treatment, which comprises incubation time and duration of the heating period, will vary. For example, in accordance with the biomass materials involved, the temperatures and other factors utilized in the hydrothermal treatment can affect the time period of the hydrothermal treatment. In one embodiment, a time period utilized is chosen that is effective to result in the recovery of hemicellulose in an amount of at least about 75% to 90% or more of the total hemicellulose available in the lignocellulosic biomass feedstock, while dissolving lignin in an amount of not more than about 5% of the total lignin available in the same feedstock.

The hydrothermal treatment can be carried out for a time period ranging from about 2 minutes to about 24 hours, or more if required. It has been determined that the upper end of this time period is applicable to treatment where enzymes are present, since the treatment is relatively slow and is performed under moderate conditions, such as a lower temperature and a slightly acidic or a slightly alkaline pH. Temperature and time are often interchangeable. As a general rule, higher temperatures can result in shorter periods of time.

In some embodiments, the aqueous biomass mixture is heated to the desired temperature and then immediately be allowed to cool (i.e. there is no hold of the aqueous biomass mixture at the high temperature). In other embodiments, the aqueous biomass mixture can be maintained at the desired temperature for some period of time to allow occurrence of the desired changes to the biomass feedstock. One of the most effective ways to conduct this reaction is through a countercurrent flow arrangement between solids and liquid so as to minimize the secondary reactions of monomeric sugars formed from hemicellulose hydrolysis.

The hydrothermal treatment can be carried out using a suitable combination of the above process parameters. For example, when higher temperatures are used, the hemicellulose can be extracted without the addition of acids or alkalis and/or for shorter periods of time. Combinations of parameters at the upper ends of the suitable ranges such as high temperatures for longer periods of time for liquid hot water solutions or stronger solutions of acids or alkalis are not preferred since under such combinations of conditions, there exists the possibility of breakdown of the lignin content of the lignocellulosic material which is not desirable. Such combinations of conditions may also lead to undesirable reactions of the hemicellulose fraction producing byproducts such as furfural.

The hemicellulose can be extracted from the biomass in single or multiple steps in an aqueous solution that is heated to a temperature ranging from about 60° C. and 200° C., and at pressures sufficient to minimize boiling. This step can be conducted with or without pH control. Generally, the pH can be between 4 and 7 so as to minimize the formation of secondary reaction products such as furfural.

When enzymes are used, temperatures not higher than about 80° C. can be used. Higher temperatures within the suitable range may be used in the acid hydrolysis of hemicellulose, especially when the pH is close to neutral, such as when no acid is added to the aqueous medium.

In other embodiments, the hydrothermal and enzymatic hydrolysis treatments can occur simultaneously in Stage 1. For example, a multistep program of hydrothermal treatment can incorporate enzymes at a point in the process where the temperatures and pH are suitable for those organisms to accelerate the conversion of oligosaccharides and polysaccharides into monomeric sugar units.

The hydrothermal or enzymatic hydrolysis treatments in Stage 1 can also further include a mixing step. Any suitable mechanical devices for mixing can be used, which are known to those skilled in this art. In addition, the hydrothermal treatment can be conducted with countercurrent flow of solids and liquid as would be achieved in an inclined, screw-type reactor used in sawdust pulping in the pulp and paper industry.

In one embodiment, the organosolv treatment comprises a mixture of water and an organic solvent at selected condition parameters that include temperature, time, pressure, solvent-to-water ratio and solids-to-liquid ratio.

The solvent can comprise, but is not limited to, alcohols, organic acids and ketones. The alcohols can be selected from the group consisting of methanol, ethanol, propanol, butanol and glycol. The organic acids can be selected from the group consisting of formic acid and acetic acid. An example of a ketone can include, but is not limited to, acetone.

If the three-stage process is carried out for the production of biobutanol and organosolv lignin, then the solvent used in Stage 2 can be butanol as this can simplify the process flowsheet and thus reduce costs.

In another embodiment, the solvent-to-water ratio can be in the range from about 10% (by weight) to anhydrous solvent. In further embodiments, the solvent-to-water ratio can be in the range of about 40% (by weight) to about 60% (by weight).

In one embodiment, the temperature can be in the range of about 100° C. to about 200° C., but not exceeding 220° C. In another embodiment, the temperature can be in the range of about 120° C. to about 200° C. In yet a further embodiment, the temperature can be in the range of about 140° C. to about 180° C.

As the hemicellulose component has been substantially removed in Stage 1, it is noted that this organosolv treatment can be less severe and, therefore, the reaction time and/or temperature can be lower than for prior art systems. This is likely due to the higher accessibility of the solvent to both the lignin and cellulose structures because of the absence of much of the hemicellulose polymer in the biomass structure.

In one embodiment, the time period for the organosolv treatment can be in the range of about 10 minutes to several hours.

In one embodiment, the organosolv treatment can be carried out in the presence of a catalyst. Catalysts that may be used can include inorganic and organic acids such as sulphuric acid, hydrochloric acid and acetic acid. Alkalis can also be used as catalysts, such as sodium hydroxide. In addition, neutral alkali earth metals such as sodium, magnesium, and aluminum salts can also be used.

In other embodiments, the organosolv treatment can also be autocatalyzed so that a catalyst can be produced naturally during the treatment and, therefore, the addition of an external catalyst is not necessary. For example, lignin solubilization during the organosolv treatment can be catalyzed by acetic acid that is naturally released from the remaining hemicellulose fraction in the first solid phase from Stage 1. The amount of acetic acid produced in such a manner in the present case may be less than required for catalysis since the hemicellulose fraction has been substantially removed from the biomass. If that is the case, then addition of acetic acid or a recycling of an acetic acid-bearing waste stream to the Stage 2 may be practiced.

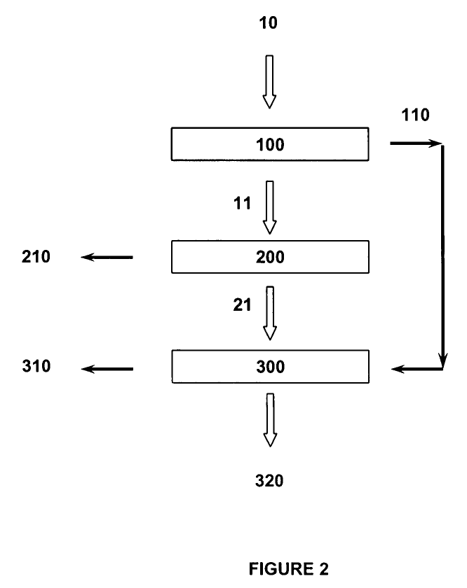

A block diagram illustrating an embodiment of the process including sequential separation to produce solid phases and liquid phases in three stages is shown in FIG. 2. Stage 1 comprises treating lignocellulosic biomass 10 by subjecting it to a series of steps as part of hydrothermal treatment 100 containing an aqueous environment at a pH of between pH 4 and pH 9, a temperature from about 40° C. to about 220° C., a pressure sufficient to maintain essentially a liquid aqueous phase and for a time period ranging from about 2 minutes to about 120 minutes.

The pH can be adjusted and maintained by adding an acid selected from the group consisting of a sulphuric acid, nitric acid, hydrochloric acid, phosphoric acid and acetic acid.

Alternatively, the pH can be adjusted by adding an alkali selected from the group consisting of a sodium hydroxide, a potassium hydroxide and a sodium carbonate.

In another embodiment, hydrothermal treatment 100 can be performed in the presence of an enzyme selected from the group consisting of a ferulic acid esterase, a xylanase and an arabinase.

In another embodiment, the biomass can be subjected to a pretreatment, such as mixing, prior to the hydrothermal treatment. The mixing can include mechanical disruption of the biomass such as by refining, grinding, cutting, chopping, or pulverizing. The pretreatment can also include a steam exposure lasting no more than 5 to 30 seconds to open up the pores of the lignocellulosic biomass.

Referring to FIG. 2, hydrothermal treatment 100 produces first solid phase 11 and first liquid phase 110, which are subjected to liquid-solid separation. First liquid phase 110 comprises hemicellulose and/or hemicellulose-derived sugars, which can be further processed if desired in accordance with established techniques known to those skilled in the art.

First solid phase 11 can be subjected to organosolv treatment 200 as part of Stage 2. The treatment medium can contain a mixture of water and an organic solvent selected from the group consisting of a lower aliphatic alcohol and a lower aliphatic carboxylic acid. Organosolv treatment 200 produces second liquid phase 210 comprising lignin and some dissolved sugars, and second solid phase 21 consisting of mostly cellulose. The dissolved sugars may be further processed if desired using conventional techniques. The solvents added in the organosolv treatment may be recovered and/or recycled back for use in the organosolv treatment using any suitable technique known to those skilled in the art, such as flash evaporation and distillation.

Second solid phase 21 is separated from the liquid stream 210 using techniques previously discussed and then transported to Stage 3 (300) which consists of the use of cellulase enzymes to convert cellulose into monomeric glucose sugar units. The monomeric glucose sugars can then be fermented with appropriate yeast and/or recombinant organisms to produce a biofuel and/or a biochemical. In one embodiment, the six-carbon glucose sugar units can be converted to bioethanol or biobutanol or a combination thereof and contained as part of aqueous stream 320. In another embodiment, the sugars are converted to 1,3 propanediol or other chemical building blocks and contained in aqueous stream 320.

Process step 300 may also allow for first liquid phase 110 from Stage 1 to be combined with second solid phase 21 in a single reactor for the purpose of conducting simultaneous saccharification (of cellulose to glucose sugar using cellulose enzymes) and co-fermentation of hemicellulose-derived monomeric sugar and said glucose units.

The fuel or chemical product contained in process stream 320 can then be separated from the aqueous stream by distillation, membrane separation, multiple effect evaporators and the like. During fermentation, yeast and/or recombinant organisms generally produce carbon dioxide gas 310 as part of the reaction mechanism and this gas 310 is vented to atmosphere or captured and purified for sale.

The separation of solids from liquids can be accomplished using any type of liquid-solid separation technique known to those skilled in this art. Those available in biomass and fiber processing can be used for separation purposes, such as filtration and centrifugation.

As evident from the above, the present process can be adapted for batch processing, continuous processing or semi-continuous processing procedures.

For example, in batch processing, hydrothermal treatment 100 and organosolv treatment 200 can be performed in a single reactor or in separate reactors. The biomass feedstock can be mixed with a sufficient amount of liquor, which can contain water or a mixture of water and an organic solvent, corresponding respectively to the hydrothermal treatment or organosolv treatment being carried out. The liquor can be maintained at the desired pH and temperature, for the desired period of time. Upon completion, liquid-solid phase separation can be carried out to recover hemicellulose, lignin and cellulose.

In continuous processing, hydrothermal treatment 100 and organosolv treatment 200 can be performed in a single reactor having two reaction zones, or in separate reactors. Biomass can be fed into the reactor in one direction while the liquor flows in the opposite direction. This countercurrent flow is well known to those skilled in the art.

In semi-continuous processing, the biomass feedstock can be packed in a column reactor, which can be heated. In the first stage, liquor containing an aqueous solution for the hydrothermal treatment can be preheated prior to being pumped into the reactor. The liquor for the hydrothermal treatment can be allowed to contact the biomass for the desired period of time to produce a hermicellulose-rich stream. In the second stage, liquor containing water and organic solvent for the organosolv treatment can be preheated and then introduced into the reactor to produce a lignin-rich stream. The hemicellulose-rich and lignin-rich streams can be recovered separately. Cellulose can be recovered from solid residues collected in the reactor.

An example of how Stages 1, 2 and 3 of the process described herein can be carried out using wheat straw as a source of biomass is set out below.

In Stage 1, one kilogram of wheat straw with an average length of 2.5 cm can be added to a two-step countercurrent pretreatment (with radial mixing of the solids throughout the length of reactor) using liquid hot water with pH maintained in the 5-7 range by addition of a small amount of sodium hydroxide. The solids concentration can be maintained at around 20 percent. The first step includes increasing the temperature of the mixture to between 80-160° C. and the residence time is around 60 minutes. The second step includes increasing the temperature to between 180-200° C. and the residence time is kept below 30 minutes. The hemicellulose dissolution can generally be found between 80-90 percent with the lignin dissolution generally less than 10% by weight.

In Stage 2, the solids from Stage 1 can be placed in a one-stage organosolv screw-type reactor with countercurrent flow of a 40% w/w ethanol/water mixture kept at a temperature of about 180° C. with radial mixing of the solids throughout the length of the reactor shaft. A small amount of acetic acid can be added to catalyze the reactions. Over 75% w/w of the starting lignin material can be solubilized by this treatment.

In Stage 3, the solids from Stage 2 can be hydrolyzed in a batch reactor with cellulase enzymes supplemented with beta-glucosidase for a period of 72 hours to produce glucose sugar monomers. The fermentation of glucose can be carried out with S. cerevisiae strain for a period of 7 days. The reactivity of the solids containing cellulose is generally found to be above 85% conversion to bioethanol.

Definitions

As used herein, the term “crude plant biomass material” and variations thereof refers to plant biomass, which has not been subjected to processing steps to remove hemicellulose or lignin. It is believed that crude plant biomass materials possess a self-buffering capacity.

As used herein, the term “aqueous biomass mixture” refers to the addition of water to biomass to place the biomass in an aqueous environment and also refers to biomass having enough moisture content of its own such that it is not necessary to add water to the biomass to produce an aqueous biomass mixture.

As used herein, the term “batch process” refers to a process wherein a material is placed in a vessel at the start and (only) removed at the end. No material is exchanged with the surroundings during the process.

As used herein, the term “continuous process” refers to a process wherein the material flows into and out of the process during the entire duration.

As used herein, the term “catalyst” refers to a chemical substance, usually used in small amounts relative to the biomass feedstock that modifies or increases the rate of the chemical reaction of the biomass feedstock, without being consumed in the process.

As used herein, the term “hydrothermal treatment” refers to the use of heated liquid water to treat biomass. When control of pH is required it is accomplished by the addition of an acid or base.

It will be noted that the present invention is one well adapted to attain all the ends and objects hereinabove set forth together with other advantages which are obvious and which are inherent to the disclosed process. Many embodiments may be made of the invention without departing from the scope thereof. Accordingly, it is to be understood that all matter herein set forth is to be interpreted as illustrative. Certain features and subcombinations that are of utility may be employed including substitutions, modifications, and optimizations, as would be available expedients to those of ordinary skill in the art.

### REFERENCES

1) Mosier, N., et al., Bioresource Technology, 96 (2005), 673-686.
2) Laser, M., et al., Bioresource Technology, 81 (2002), 33-44.
3) Larsen et al., Integration of a Biorefinery Working at a High Dry Solids Matter Content with a Power Plant. Concepts and Feasibility., 28^{th} Symposium on Biofuels and Biochemicals, (2006).
4) Chen and Liu, Bioresource Technology, 98 (2007), 666-676.
5) Pan, X., et al., Biotechnology and Bioengineering, 94(5), (2006), 851-861.
6) Laser, M., Hydrothermal Pretreatment of Cellulosic Biomass for Bioconversion to Ethanol, PhD Thesis, Dartmouth College (2001)

We claim:

1. A method for processing biomass to separately recover hemicellulose sugars, lignin and cellulose, the method comprising the steps of:
a) placing biomass in an aqueous environment to form an aqueous biomass mixture;
b) applying a sufficient amount of heat to the aqueous biomass mixture for a predetermined period of time so as to cause separation of hemicellulose from the biomass and solubilization of the hemicellulose to produce a first liquid phase containing hemicellulose sugars, and a first solid phase;
c) separating the first liquid phase from the first solid phase;
d) applying a mixture of water and at least one organic solvent to the first solid phase at a predetermined temperature so as to cause separation of lignin from the first solid phase and solubilization of the lignin to produce a second liquid phase containing lignin, and a second solid phase containing cellulose;
e) separating the second liquid phase from the second solid phase; and
f) recovering cellulose from the second solid phase.

2. The method as set forth in claim 1 further comprising the step of mixing the biomass prior to or during the application of heat to the aqueous biomass mixture.

3. The method as set forth in claim 1 wherein the aqueous biomass mixture comprises a pH of less than or approximately equal to 9.

4. The method as set forth in claim 1 wherein the step of applying heat further comprises heating the aqueous biomass mixture to a temperature in the range of about 40° C. to about 220° C.

5. The method as set forth in claim 1 wherein the step of applying heat further comprises heating the aqueous biomass mixture for a period of time in the range of about 2 minutes to about 24 hours.

6. The method as set forth in claim 1 further comprising the step of adjusting the pH by adding an acid selected from the group consisting of sulphuric acid, nitric acid, hydrochloric acid, phosphoric acid and acetic acid.

7. The method as set forth in claim 1 further comprising the step of adjusting the pH by adding an alkali selected from the group consisting of sodium hydroxide, potassium hydroxide and sodium carbonate.

8. The method as set forth in claim 1 further comprising exposing the biomass to an enzyme prior to or during the application of heat to the aqueous biomass mixture, the enzyme selected from the group consisting of ferulic acid esterase, xylanase and arabinase.

9. The method as set forth in claim 1 wherein the at least one organic solvent is selected from the group consisting of a lower aliphatic alcohol and a lower aliphatic carboxylic acid.

10. The method as set forth in claim 1 further comprising the step of precipitating lignin in a solid form from the second liquid phase.

11. The method as set forth in claim 1 further comprising the step of exposing the second solid phase to enzymatic hydrolysis and fermentation to produce biofuel and/or biochemicals.

12. The method as set forth in claim 1 further comprising the step of combining the first liquid phase and the second solid phase to result in a mixture for saccharification and fermentation.

13. The method as set forth in claim 1 further comprising the step of fermenting the first liquid phase to produce alcohol.

14. A process for separately recovering hemicellulose sugars, lignin and cellulose from biomass, the process comprising the steps of:
a) subjecting lignocellulosic biomass to at least one hydrothermal treatment for producing a first liquid phase containing hemicellulose sugars, and a first solid phase;
b) separating the first liquid phase from the first solid phase;
c) subjecting the first solid phase to an organosolv treatment for producing a second liquid phase containing lignin, and a second solid phase containing cellulose; and
d) separating the second liquid phase from the second solid phase.

15. The process as set forth in claim 14 further comprising the step of mixing the biomass prior to or during the hydrothermal treatment.

16. The process as set forth in claim 14 wherein the at least one hydrothermal treatment further comprises an aqueous environment at a pH of less than 10.

17. The process as set forth in claim 14 wherein the hydrothermal treatment further comprises the step of heating the biomass to a temperature in the range of about 40° C. to about 220° C.

18. The process as set forth in claim 14 wherein the biomass is subjected to the hydrothermal treatment for a period of time ranging from about 2 minutes to about 24 hours.

19. The process as set forth in claim 16 wherein the pH is adjusted by adding an acid selected from the group consisting of sulphuric acid, nitric acid, hydrochloric acid, phosphoric acid and acetic acid.

20. The process as set forth in claim 16 wherein the pH is adjusted by adding an alkali selected from the group consisting of sodium hydroxide, potassium hydroxide and sodium carbonate.

21. The process as set forth in claim 14 wherein the hydrothermal treatment is performed in the presence of an enzyme selected from the group consisting of ferulic acid esterase, xylanase and arabinase.

22. The process as set forth in claim 14 wherein the organosolv treatment comprises a mixture of water and at least one organic solvent selected from the group consisting of a lower aliphatic alcohol and a lower aliphatic carboxylic acid.

23. The process as set forth in claim 22 wherein the process further comprises a batch process.

24. The process as set forth in claim 22 wherein the process further comprises a continuous process.

25. A method for separately recovering hemicellulose sugars, lignin and cellulose from biomass, the method comprising the steps of:
a) placing biomass in an aqueous environment to form an aqueous biomass mixture;
b) separating a first solid phase and a first liquid phase containing hemicellulose sugars from the aqueous biomass mixture;
c) separating a second solid phase containing cellulose and a second liquid phase containing lignin from the first solid phase; and
d) recovering cellulose from the second solid phase.

26. A method for separately recovering hemicellulose sugars, lignin and cellulose from an aqueous biomass mixture, the method comprising the steps of:
a) separating a first solid phase and a first liquid phase containing hemicellulose sugars from the aqueous biomass mixture;
b) separating a second solid phase containing cellulose and a second liquid phase containing lignin from the first solid phase; and
c) recovering cellulose from the second solid phase.
